(19) United States
(12) Patent Application Publication
(10) Pub. No.: US 2022/0218804 A1
(43) Pub. Date: Jul. 14, 2022

(54) Composition comprising vitamin A and non digestible oligosaccharides

(71) Applicant: N.V. Nutricia, Zoetermeer (NL)

(72) Inventors: Linette Eustachia Maria Willemsen, Utrecht (NL); Johan Garssen, Utrecht (NL); Leon Matthieu Johannes Knippels, Utrecht (NL)

(73) Assignee: N.V. Nutricia, Zoetermeer (NL)

(21) Appl. No.: 17/611,197

(22) PCT Filed: May 18, 2020
(86) PCT No.: PCT/EP2020/063787

§ 371 Date: Nov. 15, 2021

(30) Foreign Application Priority Data
May 17, 2019 (EP) 19175096.7

(51) Int. Cl. A61K 39/00; A23L 33/155; A23L 33/21; A23L 33/00; A23L 33/18; A61K 31/07; A61K 47/26; A61K 35/20; A61K 39/35; A61P 37/02

(52) U.S. Cl. CPC A61K 39/001; A23L 33/155; A23L 33/21; A23L 33/40; A23L 33/18; A23V 2002/00; A61K 47/26; A61K 35/20; A61K 39/35; A61P 37/02; A61K 31/07

(57) ABSTRACT

The present invention relates to a combination of Vitamin A and non-digestible oligosaccharides for use in treating and/or preventing allergy.

### FIELD OF THE INVENTION

This invention relates to nutritional compositions for use in preventing and/or treating allergy in infants or young children.

### BACKGROUND OF THE INVENTION

One of the most common food allergies is cow's milk allergy (CMA). CMA affects 3-5% of infants worldwide and typically 90% of these infants outgrow their allergy by developing oral tolerance. Upon allergy persistence the only form of treatment is avoiding the causative allergen completely, although allergen specific immunotherapies for food allergy are under development. A healthy immune balance, a tightly regulated intestinal epithelial barrier function, and a well-balanced intestinal microbiome are required to acquire oral tolerance and avoid an allergic reaction towards food proteins.

Special formulae have been designed for infants that are allergic or are at increased risk of becoming allergic, for instance based on family history of atopy. Such formulae typically are hypoallergenic or allergen free and contain hydrolysed protein, with varying degrees of hydrolysis, or free amino acids. Also components like long chain polyunsaturated fatty acids may help to improve the immune system and prevent allergy. The presence of prebiotics, in particular addition of a mixture of non-digestible oligosaccharides, is known to decrease allergic symptoms in a murine model for CMA (Schouten et al. J Nutr. 2010; 140(4):835-41). Early dietary intervention with a mixture of galacto-oligosaccharides and fructo-oligosaccharides reduced the incidence of allergic manifestations and infections during the first two years of life in infants (Arslanoglu et al, 2008, J Nutr. 138 (6), p 1091-1095). EP1927292 discloses the use of a mixture of galacto-oligosaccharides and polyfructose for treatment or prevention of allergy, eczema or atopic diseases.

Vitamin A is essential for growth and development, preservation of vision and for mucosal immunity. Both Vitamin A deficiency as well as excess of Vitamin A can lead to severe problems, like blindness or toxicity. When 9cis retinoic acid (a metabolite of Vitamin A) was provided together with an ovalbumin (OVA) challenge in a BALB/c OVA allergy mouse model a reduction of OVA-IgE was found, but an increase in IgA (Heine et al, 2018, J Allerg Clin Immunol 141(2) 650-658. On the other hand, Schuster et al showed that Vitamin A deficiency may lead to a diminished asthma reaction, whereas high-level dietary vitamin A enhanced the development of experimental asthma in this model system (Schuster et al, 2008, J Immunol, 180(3) 1834-1842).

US2013/165374A1 describes a nutritional composition comprising a partially hydrolysed milk protein with a degree of hydrolysis between 15 and 25% and 50-1000 nanograms TGF-beta per 100 ml of a ready-to-consume composition, with an aim to primary prevention of allergic reactions to newly introduced dietary protein at the weaning stages. The composition may be in the form of an infant or follow-on formula, and may include minerals, vitamins and other nutrients which are understood to be essential in the daily diet. US2015/237902A1 describes infant formula specifically designed for covering nutrition necessities of infants between 0 and 36 months of life, containing all the vitamins and minerals in the required quantities in order to guarantee an adequate development of the infant. WO2018/024440A1 relates to a mix of oligosaccharides and optionally Bifidobacterium lactis for preventing, treating or reducing the severity of non-rotavirus-associated diarrhea. Infant formulas with vitamins and minerals and essential amino acids are exemplified. No active role is associated with vitamin A.

WO2019/038668A1 relates to a method and composition with human milk oligosaccharides for inducing allergen tolerance. The composition may include a vitamin A source.

Wagner Stefanie et al. “Binding of the active vitamin A metabolite retinoic acid to the major cows milk allergen bos d 5 down-regulates T-cell responses” J. Allergy and Clinical Immunology vol. 137 no. 2 (2016) investigate whether Bos d 5 could influence Th1/Th2 immune responses when complexed with the active Vitamin A metabolite retinoic acid (RA).

Still there is a need for infant formula with a further improved effect on preventing and/or treating allergy.

### SUMMARY OF THE INVENTION

Employing an animal model, the inventors found that a diet with a combination of non-digestible oligosaccharides and an increased level of dietary vitamin A was able to reduce the allergic skin response, the anaphylactic response, and the response in body temperature lowering, in mice sensitized to cow's milk protein. These effects were not observed in mice consuming a diet with a standard or increased level of vitamin A without non-digestible oligosaccharides, or a diet with non-digestible oligosaccharides and a standard dietary vitamin A level. The intestinal samples from allergic mice fed the combination of increased vitamin A and non-digestible oligosaccharides showed an increase of Ifnγ, IL10 and Foxp3 mRNA expression, and also in the spleen these mice showed an increase in CD11c⁺CD4⁻ IFNγ⁺ phagocytes.

This is indicative for increased levels of dietary Vitamin A supporting the non-digestible oligosaccharides to reduce the allergic symptom development in a mouse model for allergic sensitization, and thus of an effect of the combination of dietary vitamin A and non-digestible oligosaccharides on treating and/or preventing allergy, reducing the risk for allergy and inducing and/or enhancing oral immune tolerance to an allergen, in a subject.

### List of Clauses

clause 1. Combination of preformed Vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, for use in:
   a. treating allergy,
   b. preventing allergy,
   c. reducing the risk for allergy, and/or
   d. inducing and/or enhancing oral immune tolerance to an allergen, in a subject.
clause 2. Nutritional composition comprising 5 to 10 μg Retinol Equivalent (RE) of preformed Vitamin A per gram dry weight and/or 100 to 200 μg RE of preformed Vitamin A per 100 kcal, and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, for use in:
   a. treating allergy,
   b. preventing allergy,
   c. reducing the risk of allergy, and/or
   d. inducing/enhancing oral immune tolerance to an allergen, in a subject.
clause 3. Combination for use according to clause 1 or nutritional composition for use according to clause 2, wherein the at least one non-digestible oligosaccharide is selected from the group consisting of galacto-oligosaccharides, fructo-oligosaccharides and uronic acid oligosaccharides, more preferably from the group consisting of galacto-oligosaccharides and fructo-oligosaccharides.
clause 4. Combination for use according to clause 1 or clause 3 or nutritional composition for use according to clause 2 or clause 3, wherein the at least one non-digestible oligosaccharide is a mixture of galacto-oligosaccharides and fructo-oligosaccharides.
clause 5. Combination for use according to any one of clauses 1, 3 or 4 or nutritional composition for use according to any one of clauses 2-4, wherein said combination or composition comprises preformed Vitamin A selected from the group consisting of retinol, retinal, retinoic acid and retinyl esters, preferably retinyl esters.
clause 6. Combination for use according to any one of clauses 1, 3, 4 or 5 or nutritional composition for use according to any one of clauses 2-5, wherein the subject is an infant or young child, preferably an infant.
clause 7. Combination for use according to any one of clauses 1 or 3-6 or nutritional composition for use according to any one of clauses 2-6, wherein the subject is at risk of or suffering from a food allergy, in particular a cow's milk protein allergy.
clause 8. Nutritional composition for use according to any one of clauses 2 to 7 wherein the composition comprises 6 to 9 μg RE preformed Vitamin A per g dry weight and/or 120 to 180 μg per 100 kcal.
clause 9. Nutritional composition for use according to any one of clauses 2 to 8, wherein the composition comprises 15 to 250 mg non-digestible oligosaccharides per g dry weight.
clause 10. Nutritional composition for use according to any one of clauses 2 to 9, wherein the composition comprises a source of protein, the protein source being selected from hydrolysed protein or free amino acids, preferably hydrolysed whey protein.
clause 11. Nutritional composition for use according to any one of clauses 2 to 10, wherein the composition is an infant formula, a follow on formula or a young child formula, preferably an infant formula or a follow on formula.
clause 12. Infant formula, follow on formula or young child formula, comprising:
   i. 5 to 10 μg Retinol Equivalent (RE) preformed Vitamin A per gram dry weight and/or 100 to 200 μg RE preformed Vitamin A per 100 kcal, wherein the preformed Vitamin A is selected from the group consisting of retinol, retinal, retinoic acid and retinyl ester, preferably retinyl palmitate,
   ii. 15 to 250 mg of at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, per gram dry weight of the formula, and
   iii. hydrolysed protein and/or free amino acids, preferably hydrolysed whey protein.
clause 13. Infant formula, follow on formula or young child formula according to clause 12, wherein the at least one non-digestible oligosaccharide is selected from the group consisting of galacto-oligosaccharides, fructo-oligosaccharides and uronic acid oligosaccharides, preferably from the group consisting of galacto-oligosaccharides and fructo-oligosaccharides.
clause 14. Infant formula, follow on formula or young child formula according to clause 12 or clause 13, wherein the at least one non-digestible oligosaccharide is a mixture of galacto-oligosaccharides and fructo-oligosaccharides.
clause 15. Infant formula, follow on formula or young child formula according to any one of clauses 12-14, for use in:
   a. treating allergy,
   b. preventing allergy,
   c. reducing the risk of allergy, and/or
   d. inducing/enhancing oral immune tolerance to an allergen,
   in a subject, wherein the subject is an infant or young child that is at risk of or suffering from a food allergy, in particular a cow's milk protein allergy.

### DETAILED DESCRIPTION

The present invention relates in a first aspect to a combination of preformed Vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for use in:
a. treating allergy,
b. preventing allergy,
c. reducing the risk for allergy, and/or
d. inducing and/or enhancing oral immune tolerance to an allergen,

in a subject. The combination is preferably in the form of a nutritional composition.

In a second aspect the invention relates to a nutritional composition comprising 5 to 10 μg Retinol Equivalent (RE) of preformed Vitamin A per gram dry weight and/or 100 to 200 μg RE of preformed Vitamin A per 100 kcal, and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for use in:
a. treating allergy,
b. preventing allergy,
c. reducing the risk of allergy, and/or
d. inducing/enhancing oral immune tolerance to an allergen,

in a subject.

The invention further relates to an infant formula, a follow on formula or a young child formula, comprising:
i. 5 to 10 μg Retinol Equivalent (RE) preformed Vitamin A per gram dry weight and/or 100 to 200 μg RE of preformed Vitamin A per 100 kcal, wherein the preformed Vitamin A is selected from the group consisting of retinol, retinal, retinoic acid and retinyl ester, preferably retinyl palmitate,
ii. 15 to 250 mg of at least one non-digestible oligosaccharide selected from the group consisting of galacto-oligosaccharides, fructo-oligosaccharides, uronic acidoligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, per g dry weight of the formula, and
iii. hydrolysed protein and/or free amino acids, preferably hydrolysed whey protein.

In some jurisdictions the first aspect of the invention can be worded as the use of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for the manufacture of a medicament for (a) treating allergy, (b) preventing allergy, (c) reducing the risk for allergy, and/or (d) inducing and/or enhancing oral immune tolerance to an allergen, in a subject.

In these jurisdictions the second aspect of the invention can be worded as the use of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for the manufacture of a nutritional composition for (a) treating allergy, (b) preventing allergy, (c) reducing the risk for allergy, and/or (d) inducing and/or enhancing oral immune tolerance to an allergen, in a subject, wherein said nutritional composition comprises 5 to 10 μg Retinol Equivalent (RE) of preformed Vitamin A per gram dry weight and/or 100 to 200 μg RE of preformed Vitamin A per 100 kcal.

In some jurisdictions the first aspect of the invention can be worded as the use of a combination of preformed Vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for (a) treating allergy, (b) preventing allergy, (c) reducing the risk for allergy, and/or (d) inducing and/or enhancing oral immune tolerance to an allergen, in a subject.

In these jurisdictions the second aspect of the invention can be worded as the use of a nutritional composition comprising 5 to 10 μg Retinol Equivalent (RE) of preformed Vitamin A per gram dry weight and/or 100 to 200 μg RE of preformed Vitamin A per 100 kcal and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for (a) treating allergy, (b) preventing allergy, (c) reducing the risk of allergy, and/or (d) inducing/enhancing oral immune tolerance to an allergen, in a subject.

In some jurisdictions the first aspect of the invention can be worded as a method for treating and/or preventing allergy, reducing the risk of allergy and/or inducing and/or enhancing oral immune tolerance to an allergen in a subject, said method comprising administering a combination of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

In these jurisdictions the second aspect of the invention can be worded as a method for treating and/or preventing allergy, reducing the risk of allergy and/or inducing and/or enhancing oral immune tolerance to an allergen in a subject, said method comprising administering a nutritional composition comprising 5 to 10 μg Retinol Equivalent (RE) of preformed Vitamin A per gram dry weight and/or 100 to 200 μg RE of preformed Vitamin A per 100 kcal and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

In some jurisdictions administering a nutritional composition to an infant is considered non-therapeutic. Hence in these jurisdictions the first aspect of the invention can be worded as a non-therapeutic method for treating and/or preventing allergy, reducing the risk of allergy and/or inducing and/or enhancing oral immune tolerance to an allergen in a subject, said method comprising administering a combination of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

In these jurisdictions the second aspect of the invention can be worded as a non-therapeutic method for treating and/or preventing allergy, reducing the risk of allergy and/or inducing and/or enhancing oral immune tolerance to an allergen in a subject, said method comprising administering a nutritional composition comprising 5 to 10 μg Retinol Equivalent (RE) of preformed Vitamin A per gram dry weight and/or 100 to 200 μg RE of preformed Vitamin A per 100 kcal and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

The combination for use and the combination in the methods and uses of the invention are herein also referred to as the combination according to the invention, or the present combination.

The nutritional composition for use and the nutritional composition in the methods and uses of the invention are herein also referred to as the nutritional composition according to the invention, or the present nutritional composition.

The infant formula, follow on formula and young child formula according to the invention are herein also referred to as the formula according to the invention, or the present formula.

In the context of the present invention the term “prevention” also means “reducing the risk of” or “reducing the severity of”. The term “prevention of a certain condition” also includes “treatment of a person at risk of said condition”.

In the context of the present invention, an infant is defined as a human having an age of 0 to 12 months and a young child is defined as a human having an age of 13 to 36 months.

In the context of the present invention, the term “an infant or young child at risk of suffering from a food allergy” refers to an infant or a young child born from parents of whom one or both suffers from an atopic disease, or an infant or young child who has one or more siblings suffering from an atopic disease. These infants and young children have a higher risk of becoming allergic to certain foods, e.g. to dietary proteins, in particular cow's milk proteins.

In the context of the present invention, the term “inducing and/or enhancing oral immune tolerance to an allergen” is understood to mean that oral immune tolerance against said allergen is induced and/or enhanced, as compared to oral immune tolerance against said allergen before start of administering the combination or nutritional composition according to the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Vitamin A

The present composition contains preformed Vitamin A. Vitamin A is a fat-soluble vitamin which helps maintain normal reproduction, vision and immune function. It comes in several forms (as retinol, retinal, retinoic acid or retinyl ester). Preformed vitamin A is found only in animal-derived foods, whereas dietary carotenoids are found primarily in oils, fruits and vegetables.

Vitamin A intakes or requirements are generally expressed in terms of retinol equivalents (RE). One RE is defined as the biological activity associated with 1 μg of all-trans retinol. Hence 1 μg Retinol Equivalent is similar to 1 μg of all-trans retinol. 1 International Unit (IU) retinol corresponds to 0.3 μg Retinol Equivalents.

Currently marketed infant formula, follow on formula and young child formula with non-digestible oligosaccharides typically have Vitamin A levels of about 75 to 88 μg RE/100 kcal. According to the directives the minimum amounts of Vitamin μg RE that need to be present per 100 kcal is 60 μg RE/100 kcal (for infant formula and follow on formula).

In the present combination, the present nutritional composition and the present formula, the Vitamin A is preformed Vitamin A. Preformed Vitamin A does not encompass provitamin A carotenoids such as e.g. beta-carotene. An advantage of preformed Vitamin A is that it is better absorbed by infants and young children than provitamin A is.

The preformed Vitamin A is selected from the group consisting of retinol, retinal, retinoic acid and retinyl esters, preferably from the group consisting of retinol, retinal and retinyl esters. Examples of retinyl esters include retinyl palmitate and retinyl acetate. In a particularly preferred embodiment the preformed vitamin A is a retinyl ester, for example retinyl palmitate, retinyl acetate or a mixture thereof.

The amount of preformed Vitamin A in the present nutritional composition is 5-10 μg Retinol Equivalent (RE) per gram dry weight of the nutritional composition. Preferably, the amount of preformed Vitamin A is 6-9 μg RE per gram dry weight of the nutritional composition, more preferably 6.7-9 μg RE per gram dry weight of the nutritional composition. Consequently, the amount of preformed Vitamin A in the present nutritional composition is 500-1000 μg Retinol Equivalent (RE) per 100 gram dry weight of the nutritional composition, preferably 600-900 μg RE per 100 gram dry weight, more preferably 670-900 μg RE per 100 gram dry weight of the composition.

The amount of preformed Vitamin A in the infant formula, follow on formula or young child formula according to the invention, i.e. in the present formula, is 5-10 μg Retinol Equivalent (RE) per gram dry weight of the formula. Preferably, the amount of preformed Vitamin A is 6-9 μg RE per gram dry weight of the formula, more preferably 6.7-9 μg RE per gram dry weight of the formula. The amount of preformed Vitamin A in the present formula is thus 500-1000 μg Retinol Equivalent (RE) per 100 gram dry weight of the formula, preferably 600-900 μg RE per 100 gram dry weight of the formula, more preferably 670-900 μg RE per 100 gram dry weight of the formula.

Preferably, the present nutritional composition and the present formula comprise 100-200 μg RE preformed Vitamin A per 100 kcal, more preferably 120-180 μg RE per 100 kcal and most preferably 140-180 μg RE per 100 kcal.

Preferably, the present nutritional composition and the present formula comprise 67-135 μg RE preformed Vitamin A per 100 ml, more preferably 80-120 μg RE per 100 ml and most preferably 90-120 μg RE per 100 ml.

The amount of preformed Vitamin A in the present nutritional composition or the present formula may thus be expressed in μg RE per gram dry weight, in μg RE per 100 g dry weight, in μg RE per 100 kcal or in μg RE per 100 ml.

The nutritional composition and the infant formula, follow on formula or young child formula according to the invention comprise 5 to 10 μg RE of preformed Vitamin A per gram dry weight and/or 100 to 200 μg RE of preformed Vitamin A per 100 kcal. Preferably, the present composition and the present formula comprise 6 to 9 μg RE of preformed Vitamin A per gram dry weight and/or 120 to 180 μg RE of preformed Vitamin A per 100 kcal, more preferably 6.7 to 9 μg RE of preformed Vitamin A per gram dry weight and/or 140 to 180 μg RE of preformed Vitamin A per 100 kcal. It is to be understood that when the amount of preformed Vitamin A in the present composition and the present formula is defined as e.g. 5 to 10 μg RE of preformed Vitamin A per gram dry weight and/or 100 to 200 μg RE of preformed Vitamin A per 100 kcal, this means that the amount of preformed Vitamin A in said composition and formula can be either 5 to 10 μg RE per gram dry weight, or 100 to 200 μg RE per 100 kcal, or both.

Non-Digestible Oligosaccharides

The combination, the nutritional composition and the formula according to the present invention comprise at least one non-digestible oligosaccharide (NDO) selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides.

Advantageously and most preferred, the non-digestible oligosaccharide is water-soluble (according to the method disclosed in L. Prosky et al, J. Assoc. Anal. Chem 71: 1017-1023, 1988) and is preferably an oligosaccharide with a degree of polymerisation (DP) of 2 to 200. The average DP of the non-digestible oligosaccharide is preferably below 200, more preferably below 100, even more preferably below 60, most preferably below 40.

The non-digestible oligosaccharide is preferably a prebiotic. It is not digested in the intestine by the action of digestive enzymes present in the human upper digestive tract (small intestine and stomach). The non-digestible oligosaccharide is fermented by the human intestinal microbiota. For example, glucose, fructose, galactose, sucrose, lactose, maltose and the maltodextrins are considered digestible. The non-digestible oligosaccharide raw materials may comprise monosaccharides such as glucose, fructose, fucose, galactose, rhamnose, xylose, glucuronic acid, GalNac etc., but these are not part of the non-digestible oligosaccharides.

A suitable type of non-digestible oligosaccharide is galacto-oligosaccharides (GOS). The galacto-oligosaccharides are preferably selected from the group consisting of betagalacto-oligosaccharides, alphagalacto-oligosaccharides and galactan. Preferably the galacto-oligosaccharides are betagalacto-oligosaccharides, preferably trans-galacto-oligosaccharides. Preferably the galacto-oligosaccharides comprise galacto-oligosaccharides with beta(1,4), beta(1,3) and/or beta(1,6) glycosidic bonds and a terminal glucose. The galacto-oligosaccharides preferably have an average degree of polymerisation (DP) in the range of 2-8, preferably 3-7, i.e. are short-chain (sc) oligosaccharides in the context of the invention. Transgalacto-oligosaccharides is for example available under the trade name Vivinal®GOS (Domo FrieslandCampina Ingredients), Bi2muno (Clasado), Cup-oligo (Nissin Sugar) and Oligomate55 (Yakult). It is preferred that the non-digestible oligosaccharides in the present combination, the present nutritional composition and the present infant formula, follow on formula and young child formula at least comprises galacto-oligosaccharides as it is believed this oligosaccharide has a superior effect in allergy treatment or prevention.

Another suitable type of non-digestible oligosaccharides is fructo-oligosaccharides. Fructo-oligosaccharides may be short chain (sc) or long chain (Ic) fructo-oligosaccharides, herein also referred to as scFOS and IcFOS, respectively. Fructo-oligosaccharides may have names like fructopolysaccharides, oligofructose, polyfructose, polyfructan, inulin, levan and fructan and may refer to oligosaccharides comprising beta-linked fructose units, which are preferably linked by beta(2,1) and/or beta(2,6) glycosidic linkages, and a preferable DP between 2 and 200. Preferably, the fructo-oligosaccharides contain a terminal beta(2,1) glycosidic linked glucose.

Short chain fructo-oligosaccharides (scFOS) have an average DP below 10. Preferably the average DP is in the range of 2-8, more preferably in the range of 3-7. An example of scFOS is inulin hydrolysate products. scFOS products are for instance commercially available as Raftilose P95 (Orafti) or with Cosucra.

In long chain fructo-oligosaccharides (IcFOS) the fructo-oligosaccharides preferably contain at least 7 fructose units, preferably beta-linked. The average DP of IcFOS is preferably above 10, typically in the range of 10-100, preferably 15-50, most preferably above 20. An example of IcFOS is inulin. Inulin is a type of fructo-oligosaccharides wherein at least 75% of the glycosidic linkages are beta(2,1) linkages. Typically, inulin has an average chain length between 8 and 60 monosaccharide units. A suitable IcFOS product is commercially available under the trade name Raftiline®HP (Orafti). Other suitable sources are Raftilose (Orafti), Fibrulose and Fibruline (Cosucra) and Frutafit and Frutalose (Sensus).

Another suitable type of non-digestible oligosaccharides is uronic acid oligosaccharides. The term uronic acid oligosaccharide as used in the present invention refers to an oligosaccharide wherein at least 50 number % of the monosaccharide units present in the oligosaccharide is one selected from the group consisting of guluronic acid, mannuronic acid, galacturonic acid, iduronic acid, riburonic acid and glucuronic acid. In a preferred embodiment the uronic acid oligosaccharide comprises at least 50 number % galacturonic acid based on total uronic acid units in the uronic acid oligosaccharide. The uronic acid oligosaccharides used in the invention are preferably prepared from degradation of pectin, pectate, alginate, chondroitine, hyaluronic acids, heparine, heparane, bacterial carbohydrates, and/or sialoglycans, more preferably of pectin and/or alginate, even more preferably of pectin, most preferably of polygalacturonic acid. Preferably the degraded pectin is prepared by hydrolysis and/or beta-elimination of fruit and/or vegetable pectins, more preferably apple, citrus and/or sugar beet pectin, even more preferably apple, citrus and/or sugar beet pectin degraded by at least one lyase.

Preferably, the uronic acid non-digestible oligosaccharides have a DP of 2 to 250, more preferably a DP of 2 to 100, even more preferably a DP of 2 to 50, most preferably a DP of 2 to 20.

Preferably, the uronic acid oligosaccharide, if present, is a pectin degradation product. More preferably the uronic acid oligosaccharide, if present, is galacturonic acid oligosaccharide.

Preferably, at least one of the terminal hexuronic acid units of the uronic acid oligosaccharide has a double bond. The double bond effectively protects against attachment of pathogenic bacteria to intestinal epithelial cells, which is advantageous for infants. Preferably, one of the terminal hexuronic acid units comprises the C4-C5 double bond. The double bond at terminal hexuronic acid unit for example be obtained by enzymatic hydrolysis of pectin with lyase.

The uronic acid oligosaccharide may be derivatised. The uronic acid oligosaccharide may be methoxylated and/or amidated. If this is the case, it is preferred that the uronic acid oligosaccharides are characterized by a degree of methoxylation above 20%, preferably above 50%, even more preferably above 70%. As used herein, “degree of methoxylation” (also referred to as DE or “degree of esterification”) is intended to mean the extent to which free carboxylic acid groups contained in the uronic acid oligosaccharide have been esterified, e.g. by methylation.

The present combination, the present nutritional composition and the present formula according to the invention preferably comprises a mixture of two or more types of non-digestible oligosaccharides. It is further preferred that the two or more non-digestible oligosaccharides are selected from the group consisting of galacto-oligosaccharides, fructo-oligosaccharides and uronic acid oligosaccharides. The present invention is based on the finding that increased levels of dietary Vitamin A support non-digestible oligosaccharides in reducing the allergic symptom development. This effect is deemed similar with or without the presence of uronic acid oligosaccharides. Therefore, preferably the two or more types of non-digestible oligosaccharides are selected from the group consisting of galacto-oligosaccharides and fructo-oligosaccharides. It is particularly preferred that the present combination, the present nutritional composition and the present formula comprise two types of non-digestible oligosaccharides selected from the group consisting of galacto-oligosaccharides and fructo-oligosaccharides.

In one embodiment the invention thus relates to a combination of preformed Vitamin A and two or more non-digestible oligosaccharides selected from the group consisting of galacto-oligosaccharides, fructo-oligosaccharides and uronic acid oligosaccharides, for use in:
a. treating allergy,
b. preventing allergy,
c. reducing the risk for allergy, and/or
d. inducing and/or enhancing oral immune tolerance to an allergen,

in a subject.

In this embodiment it is further preferred that said combination comprises two types of non-digestible oligosaccharides selected from the group consisting of galacto-oligosaccharides and fructo-oligosaccharides. In one embodiment the two or more non-digestible oligosaccharides is a mixture of galacto-oligosaccharides and fructo-oligosaccharides. In another embodiment the two or more non-digestible oligosaccharides is a mixture of galacto-oligosaccharides, fructo-oligosaccharides and uronic acid oligosaccharides.

In another embodiment the invention relates to a nutritional composition comprising 5 to 10 μg Retinol Equivalent of preformed Vitamin A per gram dry weight and/or 100 to 200 μg RE of preformed Vitamin A per 100 kcal, and two or more non-digestible oligosaccharides selected from the group consisting of galacto-oligosaccharides, fructo-oligosaccharides and uronic acid oligosaccharides, for use in:
a. treating allergy,
b. preventing allergy,
c. reducing the risk of allergy, and/or
d. inducing/enhancing oral immune tolerance to an allergen,

in a subject.

In this embodiment it is further preferred that said nutritional composition comprises two types of non-digestible oligosaccharides selected from the group consisting of galacto-oligosaccharides and fructo-oligosaccharides. In one embodiment the two or more non-digestible oligosaccharides is a mixture of galacto-oligosaccharides and fructo-oligosaccharides. In another embodiment the two or more non-digestible oligosaccharides is a mixture of galacto-oligosaccharides, fructo-oligosaccharides and uronic acid oligosaccharides.

In another embodiment, the invention relates to an infant formula, follow on formula or young child formula comprising:
i. 5 to 10 μg RE preformed Vitamin A per gram dry weight and/or 100 to 200 μg RE preformed Vitamin A per 100 kcal, wherein the preformed Vitamin A is selected from the group consisting of retinol, retinal, retinoic acid, and retinyl ester, preferably retinyl palmitate,
ii. 15 to 250 mg of two or more non-digestible oligosaccharide selected from the group consisting of galacto-oligosaccharides, fructo-oligosaccharides and uronic acid oligosaccharides, per g dry weight of the formula, and
iii. hydrolysed protein and/or free amino acids, preferably hydrolysed whey protein.

In this embodiment it is further preferred that said infant formula, follow on formula or young child formula comprises two types of non-digestible oligosaccharides selected from the group consisting of galacto-oligosaccharides and fructo-oligosaccharides. In one embodiment the two or more non-digestible oligosaccharides is a mixture of galacto-oligosaccharides and fructo-oligosaccharides. In another embodiment the two or more non-digestible oligosaccharides is a mixture of galacto-oligosaccharides, fructo-oligosaccharides and uronic acid oligosaccharides.

In case the non-digestible oligosaccharide comprises or consists of a mixture of two distinct oligosaccharides, one oligosaccharide may be short-chain as defined above and one oligosaccharide may be long-chain as defined above. Most preferably, short-chain oligosaccharides and long-chain oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1.

In one embodiment, the composition comprises at least two of long chain fructo-oligosaccharides, short chain fructo-oligosaccharides and galacto-oligosaccharides. Preferred mixtures include mixtures of long-chain fructo-oligosaccharides with short chain galacto-oligosaccharides and mixtures of long-chain fructo-oligosaccharides with short-chain fructo-oligosaccharides.

In a preferred embodiment the present nutritional composition or the present formula comprises a mixture of galacto-oligosaccharides and fructo-oligosaccharides. Preferably the mixture of galacto-oligosaccharides and fructo-oligosaccharides is present in a weight ratio of from 1:99 to 99:1, more preferably from 1:19 to 19:1, more preferably from 1:1 to 19:1, more preferably from 2:1 to 15:1, more preferably from 5:1 to 12:1, even more preferably from 8:1 to 10:1, even more preferably in a ratio of about 9:1. This weight ratio is particularly advantageous when the galacto-oligosaccharides have a low average DP and fructo-oligosaccharides have a relatively high DP. Most preferred in this embodiment is a mixture of galacto-oligosaccharides with an average DP below 10, preferably below 6, and fructo-oligosaccharides with an average DP above 7, preferably above 11, even more preferably above 20. In this embodiment it is further preferred that the galacto-oligosaccharides and fructo-oligosaccharides are present in a weight ratio of from 5:1 to 12:1, the galacto-oligosaccharides have an average DP below 10 and the fructo-oligosaccharides have an average DP above 11. In this embodiment it is particularly preferred that the galacto-oligosaccharides and fructo-oligosaccharides are present in a weight ratio of from 8:1 to 10:1, more preferably about 9:1, the galacto-oligosaccharides have an average DP below 6 and the fructo-oligosaccharides have an average DP above 20. In these embodiments it is further preferred that the nutritional composition comprises 0.4 to 1.2 g, preferably 0.6 to 1.0 g, more preferably 0.7 to 0.9 g of the galacto-oligosaccharides and fructo-oligosaccharides per 100 ml. It is particularly preferred that the composition comprises about 0.8 g of the galacto-oligosaccharides and long chain fructo-oligosaccharides per 100 ml.

In another preferred embodiment the present nutritional composition or the present formula comprises a mixture of short chain (sc) fructo-oligosaccharides and long chain (Ic) fructo-oligosaccharides. Preferably the mixture of short chain fructo-oligosaccharides and long chain fructo-oligosaccharides is present in a weight ratio of from 1:99 to 99:1, more preferably from 1:19 to 19:1, even more preferably from 1:10 to 19:1, more preferably from 1:5 to 15:1, more preferably from 1:1 to 10:1. Preferred is a mixture of short chain fructo-oligosaccharides with an average DP below 10, preferably below 6 and long chain fructo-oligosaccharides with an average DP above 7, preferably above 11, even more preferably above 20. In this embodiment it is particularly preferred that the short chain fructo-oligosaccharides and long chain fructo-oligosaccharides are present in a weight ratio of from 1:5 to 15:1, the short chain fructo-oligosaccharides have an average DP below 10 and the long chain fructo-oligosaccharides have an average DP above 11. In this embodiment it is particularly preferred that the galacto-oligosaccharides and fructo-oligosaccharides are present in a weight ratio of from 1:1 to 10:1, the short chain fructo-oligosaccharides have an average DP below 6 and the long chain fructo-oligosaccharides have an average DP above 20. In these embodiments it is further preferred that the nutritional composition comprises 0.4 to 1.2 g, preferably 0.6 to 1.0 g, more preferably 0.7 to 0.9 g of the long chain fructo-oligosaccharides and short chain fructo-oligosaccharides per 100 ml. It is particularly preferred that the composition comprises about 0.8 g of the long chain fructo-oligosaccharides and short chain fructo-oligosaccharides per 100 ml.

In case the present nutritional composition or the present formula further comprises uronic acid oligosaccharides, it is preferred that the composition comprises galacto-oligosaccharides, long chain fructo-oligosaccharides and uronic acid oligosaccharides, preferably in a weight ratio of (20 to 2):1:(1 to 3), more preferably in a ratio of (15 to 5):1:(1 to 3), most preferably in a ratio of 9:1:2. Also in these embodiments it is preferred that the composition comprises 0.4 to 1.2 g, preferably 0.6 to 1.0 g, more preferably 0.7 to 0.9 g of the galacto-oligosaccharides, long chain fructo-oligosaccharides and uronic acid oligosaccharides per 100 ml. It is particularly preferred that the composition comprises about 0.8 g of the galacto-oligosaccharides, long chain fructo-oligosaccharides and uronic acid oligosaccharides per 100 ml.

The present nutritional composition or the present formula may also comprise short chain fructo-oligosaccharides, long chain fructo-oligosaccharides and uronic acid oligosaccharides in a weight ratio of (20 to 2):1:(1 to 3), preferably in a ratio of (15 to 5):1: (1 to 3), most preferably in a ratio of 9:1:2. Also in these embodiments it is preferred that the composition comprises 0.4 to 1.2 g, preferably 0.6 to 1.0 g, preferably 0.7 to 0.9 g of the fructo-oligosaccharides and uronic acid oligosaccharides per 100 ml. It is particularly preferred that the composition comprises about 0.8 g of the fructo-oligosaccharides and uronic acid oligosaccharides per 100 ml.

The present nutritional composition preferably comprises 15 mg to 250 mg non-digestible oligosaccharides per gram dry weight of the composition, more preferably 25 to 150 mg per gram dry weigh, even more preferably 30 to 100 mg per gram dry weight, most preferably 50 to 75 mg per gram dry weight.

The present nutritional composition or the present formula preferably comprises 2.5 to 15 wt. % of the non-digestible oligosaccharides, based on dry weight of the formula, more preferably 3.0 to 10 wt %, and most preferably 5.0 to 7.5 wt %, all based on dry weight of the nutritional composition.

Nutritional Composition and Infant Formula, Follow on Formula and Young Child Formula

The nutritional composition according to the invention can be used as a nutritional composition, nutritional therapy, nutritional support, as a medical food, as a food for special medical purposes or as a nutritional supplement. The present nutritional composition is preferably an enteral (oral) composition. The composition is administered orally to, or intended to be administered orally to, a subject in need thereof, in particular to children and infants, including toddlers, preferably children up to 6 years of age, preferably infants or young children with an age of 0-36 months, more preferably infants 0-12 months of age, most preferably 0-6 months of age. Thus, in a preferred embodiment, the present nutritional composition is an infant formula, a follow-on formula or a young child formula (also referred to as growing-up milk), preferably it is an infant formula or follow-on formula, most preferably an infant formula. The term ‘infant formula’ is well-defined and controlled internationally and consistently by regulatory bodies. In particular, CODEX STAN 73-1981 “Standard For Infant Formula and Formulas For Special Medical Purposes Intended for Infants” is widely accepted. It recommends for nutritional value and formula composition, which require the prepared milk to contain per 100 ml not less than 60 kcal (250 kJ) and no more than 70 kcal (295 kJ) of energy. FDA and other regulatory bodies have set nutrient requirements in accordance therewith.

An infant formula is defined as a formula for use in infants and can for example be a starter formula, intended for infants of 0 to 6 or 0 to 4 months of age. A follow on formula is intended for infants of 4 or 6 months to 12 months of age. At this age infants start weaning on other food. A young child formula, or toddler or growing up milk or formula is intended for children of 12 to 36 months of age.

The formula according to the present invention is preferably an infant formula or a follow on formula, more preferably an infant formula.

Preferably, the present nutritional composition or the present formula is for providing the daily nutritional requirements to a human, in particular for administration to, in particular for feeding, humans, in particular infants including toddlers.

Preferably, the nutritional composition according to the invention, or the infant formula, follow on formula or young child formula according to the invention, comprises a protein component, a lipid component and a digestible carbohydrate component. More preferably, said nutritional composition or said formula according to the invention has the following calorie distribution: the lipid component preferably provides 30 to 60% of total calories, preferably 35 to 50% of total calories, the protein component preferably provides 5 to 20%, more preferably 5 to 15% of the total calories, in particular 6 to 12% of the total calories and the digestible carbohydrate component preferably provides 25 to 65% of the total calories, preferably 40 to 60% of the total calories. The amount of total calories is determined by the sum of calories derived from protein, lipids and digestible carbohydrates. The protein component, lipid component and digestible carbohydrate component are described in more detail below.

In order to meet the caloric requirements of the infant, the present nutritional composition or the present infant formula, follow on formula or young child formula, preferably comprises 50 to 200 kcal/100 ml liquid, more preferably 60 to 90 kcal/100 ml liquid, even more preferably 60 to 75 kcal/100 ml liquid. This caloric density ensures an optimal ratio between water and calorie consumption. The osmolarity of the present composition is preferably between 150 and 420 mOsmol/l, more preferably 260 to 320 mOsmol/l. The low osmolarity aims to reduce gastrointestinal stress.

The present nutritional composition or the present formula is preferably in a dry form in the form of a powder, in the form of a ready to feed liquid or in the form of a liquid concentrate. If the present nutritional composition or the present formula is in a dry form it is preferably in the form of a powder suitable for making a liquid composition after reconstitution with water. The present nutritional composition or the present formula can also be in a liquid concentrate form, which is diluted with water prior to use. If the present nutritional composition or the present formula is in the form of a ready to feed liquid it already contains a liquid solvent such as water and thus does not have to be reconstituted or diluted prior to use.

Preferably, when the present nutritional composition or the present formula is in a liquid form, the viscosity is below 35 mPa·s, more preferably below 6 mPa·s as measured in a Brookfield viscometer at 20° C. at a shear rate of 100 s−1.

When the present nutritional composition or the present formula is in a liquid form, the preferred volume administered on a daily basis is in the range of about 80 to 2500 ml, more preferably about 450 to 1000 ml per day.

The present nutritional composition or the present formula is not human breast milk. Preferably, the present nutritional composition or the present formula is free of living probiotics, in particular bifidobacteriae or lactobacillae. Preferably, the present nutritional composition or the present formula is free of dead probiotics, in particular bifidobacteriae or lactobacillae Since such bacteria are typically pre-cultured on milk based growth media, addition of such probiotics bears the risk of introducing traces of intact cow's milk protein. Preferably, the present nutritional, preferably enteral, composition is free of growth factors and/or cytokines. Preferably, the present nutritional, preferably enteral, composition is free of TGF, particularly TGF-beta.

Protein

Preferably, the present nutritional composition or the present infant formula, follow on formula or young child formula comprises a protein component. The protein component comprises one or more sources of protein. The terms “protein” and “protein component” encompass intact proteins, peptides, free amino acids and partially or extensively hydrolysed proteins.

The present nutritional composition or the present formula preferably comprises 4 to 25%, more preferably 5 to 20%, more preferably 7 to 16%, most preferably 7 to 12% protein, based on total calories. The present composition or the present formula, when in liquid form, preferably contains 0.5 to 6.0 g, more preferably 0.8 to 3.0 g, even more preferably 1.0 to 2.5 g of protein per 100 ml. The present composition preferably comprises at least 7.0 wt %, more preferably at least 8.0 wt %, most preferably at least 9 or at least 10 wt % protein based on dry weight of the total composition. Preferably, the present composition comprises at most 40 wt %, more preferably at most 15 wt %, preferably at most 20 wt % of protein based on dry weight of the total composition.

It is preferred that the protein component is non-allergenic or hypoallergenic. Preferably the protein component comprises free amino acids or hydrolysed protein, more preferably hydrolysed whey protein.

Infants or young children born from parents of whom one or both suffers from an atopic disease, or who have one or more siblings suffering from an atopic disease, have a higher risk of becoming allergic to dietary proteins. For these infants and young children, who are at risk of suffering from a food allergy, in particular a cow's milk allergy, it is preferred that the protein component comprises partially hydrolysed proteins (a partial protein hydrolysate), more preferably partially hydrolysed whey proteins (a partial whey protein hydrolysate). These partially hydrolysed proteins have a decreased allergenicity. Typically, the extent of hydrolysis of these partially hydrolysed proteins is less than that of extensively hydrolysed proteins for infants already suffering from allergy. These formulations have the advantage of not only reducing the risk of developing an allergic response by preventing sensitization to the protein, but also support the natural development of oral immune tolerance to the intact protein. This brings the advantage that later on the native protein can be introduced in the diet, with a reduced risk on allergic reactions.

For infants and young children suffering from an allergy to cow's milk protein, it is preferred that the protein component comprises extensively hydrolysed proteins (an extensive protein hydrolysate), more preferably extensively hydrolysed whey protein (an extensively hydrolysed whey protein hydrolysate). In extensively hydrolysed proteins hardly or no allergenic proteins or peptides are present.

For infants and young children suffering from a severe allergy to cow's milk protein, it is preferred that the protein component solely comprises free amino acids as nitrogen source.

The present nutritional composition or the present formula preferably contains at least 50 wt % protein component derived from non-human milk, more preferably at least 90 wt %, based on dry weight of total protein. It is further preferred that at least 50 wt % protein component, more preferably at least 90 wt %, based on dry weight of total protein, is derived from milk from a species of the genus Bos, Bison, Bubalus or Capra, more preferably from genus Bos, most preferably from cow's milk (Bos taurus).

The protein component of the nutritional composition or the formula according to the present invention preferably contains less than 1 wt % intact mammalian (cow)'s milk protein. More preferably the nutritional composition or the formula according to the present invention does not contain intact mammalian (cow)'s milk protein. The composition or formula may comprise an additional protein source selected from the group consisting of free amino acids and proteins from other sources such as soy, pea, rice, collagen or the like, in intact form, in partially hydrolysed form, and/or in extensively hydrolysed form.

Protein hydrolysates and whey protein hydrolysates suitable for the present nutritional composition and the present formula are known from the art. For example partial protein hydrolysates, in particular partial whey protein hydrolysates, are described in detail in WO 2011/151059, incorporated by reference herein, and extensively hydrolysed proteins are described in detail in WO 01/41581 (page 13, line 13 to page 16, line1), incorporated by reference herein.

In a preferred embodiment, the nutritional composition or the formula according to the invention comprises at least 50 wt %, more preferably at least 70 wt %, even more preferably at least 95 wt % of hydrolysed whey protein based on total protein. A suitable source of whey protein is for example a mixture of acid whey protein and demineralised sweet whey protein. Acid whey and sweet whey are commercially available. Sweet whey is the by-product of rennet-coagulated cheese and comprises caseinoglycomacropeptide (CGMP), and acid whey (also called sour whey) is the by-product of acid-coagulated cheese, and does not contain CGMP. Suitable sources for the whey protein are demineralised whey (Deminal, Friesland Campina, the Netherlands) and/or whey protein concentrate (WPC80, Friesland Campina, the Netherlands). The whey protein preferably comprises acid whey, more preferably at least 50 wt %, more preferably at least 70 wt % acid whey, based on total whey protein. Acid whey has an improved amino acid profile compared to sweet whey protein.

Hydrolysis may be achieved using a mixture of microbial endopeptidases and exopeptidases using the method as described in example 1 of WO 2011/151059, herein incorporated by reference. Preferably a mixture of an endoprotease and exoprotease is employed.

The size distribution of the peptides in the protein hydrolysate can be determined by means of size exclusion high pressure liquid chromatography as known in the art. Saint-Sauveur et al. (“Immunomodulating properties of a whey protein isolate, its enzymatic digest and peptide fractions” Int. Dairy Journal (2008) vol. 18(3), p. 260-270) describes an example thereof. In short, the total surface area of the chromatograms is integrated and separated into mass ranges expressed as percentage of the total surface area. The mass ranges are calibrated using peptides/proteins with a known molecular mass. The protein hydrolysate is preferably characterised in that it comprises between 64 and 89 wt % peptides with a molecular weight below 1000 D, between 10 and 30 wt % peptides with a molecular weight between 1000 and 5000 D and between 1 and 6 wt % of peptides or proteins with a molecular weight above 5 kD, all based on total protein.

Preferably, the nutritional composition according to the present invention, or the infant formula, follow on formula or young child formula according to the present invention, further comprises a lipid component and, as a carbohydrate component, at least one digestible carbohydrate. The lipid component and the at least one digestible carbohydrate are described in more detail below.

Lipid

The nutritional composition or the formula according to the invention preferably comprises a lipid component, preferably a lipid component suitable for infant nutrition as known in the art. The lipid component of the present nutritional composition or of the present formula preferably provides 2.9 to 6.0 g, more preferably 4 to 6 g per 100 kcal of the composition. When in liquid form, the composition preferably comprises 2.1 to 6.5 g lipid per 100 ml, more preferably 3.0 to 4.0 g per 100 ml. Based on dry weight, the present nutritional composition or the present infant formula, follow on formula or young child formula preferably comprises 12.5 to 40 wt % lipid, more preferably 19 to 30 wt %.

The present nutritional composition or the present formula preferably comprises as lipids vegetable lipids and/or marine oils, such as algae oils, bacterial oils, animal oils, vegetable oils or fish oils. Preferably, said composition or formula comprises long chain polyunsaturated fatty acids (LC-PUFA). LC-PUFA are fatty acids wherein the acyl chain has a length of 20 to 24 carbon atoms (preferably 20 or 22 carbon atoms) and wherein the acyl chain comprises at least two unsaturated bonds between said carbon atoms in the acyl chain. More preferably the present composition or present formula comprises at least one LC-PUFA selected from the group consisting of eicosapentaenoic acid (EPA, 20:5 n3), docosahexaenoic acid (DHA, 22:6 n3), arachidonic acid (ARA, 20:4 n6) and docosapentaenoic acid (DPA, 22:5 n3), preferably DHA, EPA and/or ARA. Such LC-PUFAs have a further beneficial effect on reducing the risk for allergy.

The preferred content of LC-PUFA in the present nutritional composition or present formula does not exceed 15 wt. % of total fatty acids, preferably does not exceed 10 wt. %, even more preferably does not exceed 5 wt. %. Preferably the present composition comprises at least 0.2 wt. %, preferably at least 0.25 wt. %, more preferably at least 0.35 wt. %, even more preferably at least 0.5 wt. % LC-PUFA of total fatty acids, more preferably DHA. The present composition preferably comprises ARA and DHA, wherein the weight ratio ARA/DHA preferably is above 0.25, preferably above 0.5, more preferably 0.75-2, even more preferably 0.75-1.25. The weight ratio is preferably below 20, more preferably between 0.5 and 5. The amount of DHA is preferably above 0.2 wt %, more preferably above 0.3 wt %, more preferably at least 0.35 wt %, even more preferably 0.35-0.6 wt % on total fatty acids.

Digestible Carbohydrate

Preferably, the at least one digestible carbohydrate comprises one or more digestible carbohydrates which are known in the art to be suitable for use in food products, in particular infant and young child nutritional compositions, e.g. selected from digestible polysaccharides (e.g. starch, maltodextrin), digestible monosaccharides (e.g. glucose, fructose), and digestible disaccharides (e.g. lactose, sucrose). Particularly preferred is maltodextrin and/or lactose. The use of maltodextrin is advantageous insofar that it has a higher molecular weight and can partially compensate for the increased osmolarity caused by free amino acids, if used.

In a preferred embodiment the amount of lactose is less than 40 wt % based on total digestible carbohydrates. In this embodiment it is further preferred that the present nutritional composition or the present formula does not include lactose as a digestible carbohydrate.

In another preferred embodiment, the digestible carbohydrate component preferably comprises at least 60 wt % lactose based on total digestible carbohydrate, more preferably at least 75 wt %, even more preferably at least 90 wt % lactose based on total digestible carbohydrate.

The nutritional composition or the infant formula, follow on formula or young child formula according to the invention preferably comprises other components, such as vitamins and/or minerals, preferably according to international directives for infant and follow on formulae.

The nutritional composition and the infant formula, follow on formula or young child formula in the context of the present invention are defined here above. For the avoidance of doubt it is reiterated that all embodiments apply to all aspects of the invention, including the nutritional composition for use, the nutritional composition as such, the methods and uses of the nutritional composition, the infant formula, follow on formula and young child formula as such and the infant formula, follow on formula and young child formula for use according to the invention.

Application

It was found that a combination of an enhanced level of dietary Vitamin A and non-digestible oligosaccharides was able to reduce the allergic skin response, the anaphylactic response, and the response in body temperature lowering, in mice sensitized to cow's milk protein. These effects were not observed in mice consuming a diet with standard or increased level of vitamin A alone, or a diet with non-digestible oligosaccharides and a standard dietary vitamin A level. The intestinal samples from allergic mice fed the combination of increased vitamin A and non-digestible oligosaccharides showed an increase of Ifnγ, IL10 and Foxp3 mRNA expression, and also in the spleen these mice showed an increase in CD11c⁺CD4⁻ IFNγ⁺ phagocytes. These results are indicative for increased levels of dietary Vitamin A supporting the non-digestible oligosaccharides to reduce the allergic symptom development in a mouse model for allergic sensitization, and thus of an effect of the combination of dietary vitamin A and non-digestible oligosaccharides on treating and/or preventing allergy, reducing the risk for allergy and inducing and/or enhancing oral immune tolerance to an allergen, in a subject.

Therefore the present invention relates in a first aspect to a combination of preformed Vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, for use in:
a. treating allergy,
b. preventing allergy,
c. reducing the risk for allergy, and/or
d. inducing and/or enhancing oral immune tolerance to an allergen,

in a subject.

In a second aspect the invention relates to a nutritional composition comprising 5 to 10 μg Retinol Equivalent (RE) of preformed Vitamin A per gram dry weight and/or 100 to 200 μg RE of preformed Vitamin A per 100 kcal, and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for use in:
a. treating allergy,
b. preventing allergy,
c. reducing the risk of allergy, and/or
d. inducing/enhancing oral immune tolerance to an allergen, in a subject.

The invention further relates to an infant formula, a follow on formula or a young child formula, comprising:
i. 5 to 10 μg Retinol Equivalent (RE) preformed Vitamin A per gram dry weight and/or 100 to 200 μg RE preformed Vitamin A per 100 kcal, wherein the preformed Vitamin A is selected from the group consisting of retinol, retinal, retinoic acid and retinyl ester, preferably retinyl palmitate,
ii. 15 to 250 mg of at least one non-digestible oligosaccharide selected from the group consisting of galacto-oligosaccharides, fructo-oligosaccharides, uronic acidoligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, per g dry weight of the formula, and
iii. hydrolysed protein and/or free amino acids, preferably hydrolysed whey protein, for use in:
a. treating allergy,
b. preventing allergy,
c. reducing the risk of allergy, and/or
d. inducing/enhancing oral immune tolerance to an allergen,

in a subject, wherein the subject is an infant or young child that is at risk of or suffering from a food allergy, in particular a cow's milk protein allergy.

In a further aspect, the invention relates to the use of Vitamin A for enhancing the oral tolerance induction effect, the allergy preventing effect or the allergy treating effect of a non-digestible oligosaccharide, wherein the non-digestible oligosaccharide comprises at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides.

In some jurisdictions the first aspect of the invention can be worded as the use of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for the manufacture of a medicament for (a) treating allergy, (b) preventing allergy, (c) reducing the risk for allergy, and/or (d) inducing and/or enhancing oral immune tolerance to an allergen, in a subject.

In these jurisdictions the second aspect of the invention can be worded as the use of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for the manufacture of a nutritional composition for (a) treating allergy, (b) preventing allergy, (c) reducing the risk for allergy, and/or (d) inducing and/or enhancing oral immune tolerance to an allergen, in a subject, wherein said nutritional composition comprises 5 to 10 μg Retinol Equivalent (RE) of preformed Vitamin A per gram dry weight and/or 100 to 200 μg RE of preformed Vitamin A per 100 kcal.

In some jurisdictions the first aspect of the invention can be worded as the use of a combination of preformed Vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for (a) treating allergy, (b) preventing allergy, (c) reducing the risk for allergy, and/or (d) inducing and/or enhancing oral immune tolerance to an allergen, in a subject.

In these jurisdictions the second aspect of the invention can be worded as the use of a nutritional composition comprising 5 to 10 μg Retinol Equivalent (RE) of preformed Vitamin A per gram dry weight and/or 100 to 200 μg RE preformed Vitamin A per 100 kcal and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, for (a) treating allergy, (b) preventing allergy, (c) reducing the risk of allergy, and/or (d) inducing/enhancing oral immune tolerance to an allergen, in a subject.

In some jurisdictions the first aspect of the invention can be worded as a method for treating and/or preventing allergy, reducing the risk of allergy and/or inducing and/or enhancing oral immune tolerance to an allergen in a subject, said method comprising administering a combination of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

In these jurisdictions the second aspect of the invention can be worded as a method for treating and/or preventing allergy, reducing the risk of allergy and/or inducing and/or enhancing oral immune tolerance to an allergen in a subject, said method comprising administering a nutritional composition comprising 5 to 10 μg Retinol Equivalent (RE) of preformed Vitamin A per gram dry weight and/or 100 to 200 μg RE of preformed Vitamin A per 100 kcal and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

In some jurisdictions administering a nutritional composition to an infant is considered non-therapeutic. Hence in these jurisdictions the first aspect of the invention can be worded as a non-therapeutic method for treating and/or preventing allergy, reducing the risk of allergy and/or inducing and/or enhancing oral immune tolerance to an allergen in a subject, said method comprising administering a combination of preformed vitamin A and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

In these jurisdictions the second aspect of the invention can be worded as a non-therapeutic method for treating and/or preventing allergy, reducing the risk of allergy and/or inducing and/or enhancing oral immune tolerance to an allergen in a subject, said method comprising administering a nutritional composition comprising 5 to 10 μg Retinol Equivalent (RE) of preformed Vitamin A per gram dry weight and/or 100 to 200 μg RE of preformed Vitamin A per 100 kcal and at least one non-digestible oligosaccharide selected from the group consisting of galacto-oligosaccharides, fructo-oligosaccharides, uronic acid oligosaccharides, non-digestible dextrin, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides and chito-oligosaccharides, preferably at least one non-digestible oligosaccharide selected from the group consisting of galacto-oligosaccharides and fructo-oligosaccharides, to the subject.

The methods and uses according to the present invention, comprising administering the nutritional composition or the infant formula, follow on formula or young child formula to a subject, also refer to administering an effective amount of the nutritional composition or the formula to the subject.

The allergy that is treated or prevented, or the allergy of which the risk is reduced, in the context of the present invention is preferably an allergy against dietary proteins, in particular against mammalian milk protein, more in particular an allergy against cow's milk protein.

The allergen to which the oral immune tolerance is induced and/or enhanced in the context of the present invention refers to a food allergen, in particular an allergen present in mammalian milk protein, more in particular an allergen present in cow's milk protein.

Without wishing to be bound by theory, standard levels of Vitamin A may be too low in the neonate, or in infants or young children that are at risk of developing a food allergy or that are suffering from a food allergy, and under such conditions an increased level of dietary Vitamin A was found to be effective.

In one embodiment, the subject is an infant or young child, preferably an infant with an age of 0-12 months or a young child with an age of 13-36 months. More preferably the subject is an infant having an age of 0-12 months, most preferably an infant having an age of 0-6 months.

In another embodiment, the subject is an infant that is at risk of developing a food allergy, in particular a cow's milk protein allergy. In this embodiment the nutritional composition and infant formula for use and the nutritional composition in the uses and methods of the present invention is used as a primary prevention of food allergy. An infant at risk of developing a food allergy is an infant born from parents of whom one or both suffers from an atopic disease, or an infant who has one or more siblings suffering from an atopic disease. These infants and young children have a higher risk of becoming allergic to certain foods, e.g. to dietary proteins, in particular cow's milk proteins.

In another embodiment, the subject is an infant that suffers from a food allergy, preferably a cow's milk allergy. In this embodiment the nutritional composition and infant formula for use and the nutritional composition in the uses and methods of the present invention is used as a secondary prevention of a food allergy and as a dietary management of a food allergy, preferably multiple food protein allergies and/or a cow's milk allergy.

### BRIEF DESCRIPTION OF THE FIGURES

FIG. 1. Clinical symptoms in control and whey sensitized mice fed Vitamin A and a mixture of non-digestible oligosaccharides GFA. The acute allergic skin response measured one hour after intra-dermal injection. Values are means±SEM, n=8-10, a one-way ANOVA and Bonferroni post hoc was used. Δ μm Indicates ear thickness before i.d. injection deducted from thickness after i.d. injection.

FIG. 2: The anaphylactic symptom scores measured at thirty minutes after challenge. Values are means±SEM, n=8-10. Data is calculated with the Kruskal-Wallis and Dunn's post hoc test.

FIG. 3: The body temperature was measured 30 minutes after i.d. injection via transponder read out. Values are means±SEM, n=8-10, a one-way ANOVA and Bonferroni post hoc was used. Asterix indicates a significant difference compared to sham, **** p<0.0001, *** p<0.001, * p<0.05. The ‘&’ sign indicates a significant difference compared to the VitA⁺ group. GFA, short-chain Galacto-oligosaccharides/long-chain Fructo-oligosaccharides/pectin derived Acidic-oligosaccharides; s, standard 4000 ILl/kg vitamin A; +, enriched vitamin A 8000 ILl/kg.

### EXAMPLES

### Example 1: A Diet with Non-Digestible Oligosaccharides and an Increased Level of Vitamin a Results in a Reduced Allergic Reaction in Mice Sensitized to Whey Protein

Material and methods: The semi-synthetic cow's milk free AlN93G chow (Research Diet Services, Wijk bij Duurstede, the Netherlands) was used as control diet. In all diets casein and whey were replaced by soy protein. The diets were composed, with ‘standard’ 4000 IU Vitamin A/kg or enriched with 8000 IU Vitamin A/kg (VitA⁺). For the latter supplementation, twice the standard amount of Vitamin A is chosen, for which it is known that no adverse effects occur for this dose and time frame. Before start of dietary intervention, mice were born and weened by their mothers which received standard chow (without whey or casein) containing the standard Vitamin A dose of 4000 IU/kg. Beyond Vitamin A, diets were either supplemented or not supplemented with a mixture of non-digestible oligosaccharides. One percent iso-caloric supplementation of scGOS (Vivinal GOS, Friesland Campina Domo, Zwolle, The Netherlands), IcFOS (Raftiline HP, Orafti, Wijchen, The Netherlands), and pAOS (Südzucher, Mannheim, Germany) in a 9:1:2 wt ratio was added at the expense of cellulose and lactose.

Four weeks old and body weight >11 gram, specific pathogen-free female C3H/HeOuJ mice (Charles River, Sulzfeld, Germany) were housed under conventional housing at the animal facility (Intravacc, Bilthoven, the Netherlands). Mice were held in a light/dark cycle of 12 h/12 h, controlled 65-70% relative humidity and 22±2° C. temperature with ad libitum access to tap water and pelleted food. Upon arrival, mice were fed the specific diets which was continued throughout the study: control diet (s), enriched vitamin A (VitA+) diet, diet supplemented with GFA (GFA) and diet supplemented with GFA and enriched Vitamin A (VitA⁺/GFA). Mice (n=10-15) were housed in groups and kept in two cohorts (5/cage) in Makrolon III-H cages. For cage enrichment mice were provided with two red-transparent polycarbonate igloo's on 9kGy irradiated sawdust bedding (Lignocel 9s, J. Rettenmaier & Söhne GmbH, Germany).

Following 13 days of pre-treatment with the diets (day −14 to 0), mice were sensitized weekly for five times (day 0, 7, 14, 21, 28) by means of oral gavage, with 10 μg Cholera Toxin (CT, List Biological Laboratories, USA) or 10 μg CT/20 mg whey protein concentrate (sweet whey protein concentrate 60 (sWPC60), Milei, Germany) per 500 μl Dulbecco's phosphate-buffered saline (DPBS) (Life Technologies, Inc., Invitrogen, USA) per oral gavage using a blunt needle (stainless steel) (Kent Scientific Corporation, USA). Temperature transponders (IPTT-300, BMDS, USA) were injected subcutaneously under isoflurane gas anesthesia, between the 3^{rd} and 4^{th} sensitization.

To measure the acute allergic skin response whey protein was injected intradermally (i.d.) (10 μg whey /20 μl PBS) in the ear pinnae at day 35. The acute allergic skin response was determined as delta (A) ear swelling (expressed as Δ μm) by subtraction the ear thickness measured just before i.d. injection from the ear thickness measured one hour after i.d. challenge using a digital micrometer (Mitutoyo, Veenendaal, the Netherlands). The body temperature and the anaphylactic shock symptoms were scored according to the method previously described by van Esch et al Toxicology Letters 220 (2013) 95-102.

Eighteen hours before the end of the study, mice were challenged intragastrically (i.g). with 50 mg whey/500 μl DPBS to activate the mucosal gastrointestinal immune response. At day 37 under terminal isoflurane gas anesthesia, blood and MLN were isolated and stored for further analysis.

One centimetre of the intestinal mid small intestine was collected after sacrifice and stored in RNAlater™ (Qiagen GmbH, Hilden, Germany) at 4° C. until further processing, described previously by Kerperien et al. (Pediatr Allergy Immunol. 2014; 25(8):747-54). mRNA levels were calculated with CFX Manager software (version 1.6) and corrected for the expression of Ribosomal protein S13(Rps13) with 100×2^{\hat{ }(Rps13-gene of interest)} as described previously (Garcia-Vallejo J J et al. Analytical biochemistry. 2004; 329(2):293-932). Validated primers for Il10, Ifnγ, Foxp3 were purchased from SAbioscience (Qiagen, German Town, Md., USA).

The splenocyte suspension was lysed to remove red blood cells as described previously (Kostadinova et al, Front Immunol. 2016; 7:673). MLN and spleen cell suspensions were made with a 70 μm cell strainers (Thermo Fisher Scientific, Amsterdam, the Netherlands), resuspended in RPMI 1640, 10% fetal bovine serum and penicillin (100 U/mL)/streptomycin (100 μg/mL) and quantified using a Coulter Z1 particle counter (Beckmann Coulter, Brea, USA). One million cells per well were added to polypropylene V-bottom 96 well plates (BD Biosciences, Germany) and blocked with rat anti-mouse CD16/32 (BD Biosciences, Germany) for 20 minutes at 4° C. to block non-specific binding sites. For surface staining the cells were incubated with the different antibodies of the staining panels for 30 minutes at 4° C. After the antibody incubation, the cells were fixed using BD cell fix (Becton Dickinson, Belgium). Directly after the staining procedure for extracellular markers, these cells were permeabilized using a fixation/permeabilization buffer (eBioscience, San Diego, USA) overnight. The next day, the cells were blocked again and incubated with the intracellular marker antibodies for 30 minutes at 4° C. Then the cells were fixed with the BD cell fix. Fluorescence Minus Ones (FMOs) and unstained cells were used as controls. In addition, compensation staining with UltraComp beads (eBioscience, San Diego, USA) was performed to correct for the spectral overlap, which occurs in multi-color staining. Cell surface staining antibodies used were: CD8a-APC-Cy7, CD11c-PerCP-Cy5.5 and CD25-Pe-Cy7 from BD biosciences (San Jose, Calif., USA) and CD4-PerCP-Cy5.5, CD69-Pe-Cy7, CD11b-PE, CD103-APC, CX3CR1-FITC, B220-APC from Ebiosciences (San Diego Calif., USA). Cytokines and intracellular staining for IFNγ-APC, came from Ebiosciences (San Diego Calif., USA). The analysis of the stained cells was performed using FACS Canto II cytometer (BD Biosciences, USA) and FACS Diva software (BD Biosciences, Germany).

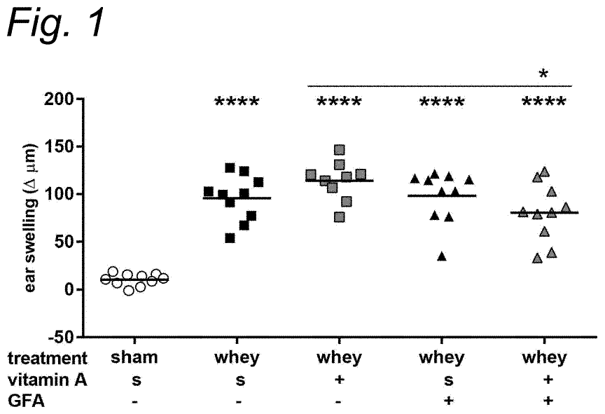

To determine the sample size historical data with sWPC60 as whey source and ear swelling of allergic mice (123±20 μm) as primary outcome parameter were used. The estimated effect size was set at 20% with 80% power and an a of 5%. Statistical analysis was performed using GraphPad Prism 7 (GraphPad Software, San Diego, Calif., USA). Not normal distributed numerical data was LOG transformed. Results are shown in FIG. 1. The data are represented as the mean±SEM plots. One-way ANOVA and post hoc Bonferroni multiple comparisons test with selected groups were used to analyze the data. Selected groups are sham vs. allergic mice (as model control), allergic mice fed a diet with standard level of Vitamin A vs. allergic mice fed a diet enriched in Vitamin A (VitA⁺,) allergic mice fed a diet with standard level of Vitamin A vs. allergic mice fed a diet with a standard level of Vitamin A and a mixture of non-digestible oligosaccharides (GFA), allergic mice fed VitA⁺ vs. allergic mice fed VitA⁺/GFA and allergic mice fed a diet with standard level of Vitamin A and GFA vs. allergic mice fed VitA⁺/GFA. Kruskal-Wallis was used to analyze the anaphylactic shock score (categorial data), followed by Dunn's multiple comparison post-test. P-value <0.05 was considered significant.

Results

The allergic symptoms measured were the acute allergic skin response, anaphylaxis and body temperature. Table 1 shows that the % anaphylactic shock was lowest in the group consuming the diet with the non-digestible oligosaccharides and enriched in Vitamin A (VitA+/GFA).

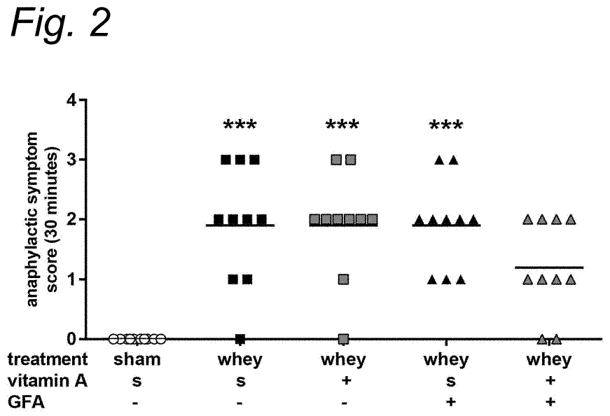

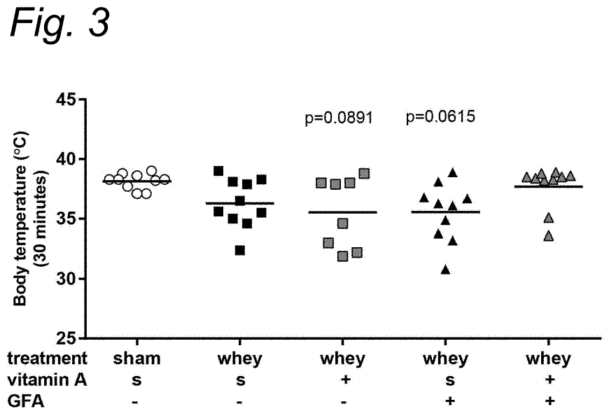

FIG. 1 shows the acute allergic skin response measured one hour after intra-dermal injection. Values are means±SEM, n=8-10, a one-way ANOVA and Bonferroni post hoc was used. Δ μm Indicates ear thickness before i.d. injection deducted from thickness after i.d. injection. FIG. 2 shows the anaphylactic symptom scores measured at thirty minutes after challenge. Values are means±SEM, n=8-10. Data is calculated with the Kruskal-Wallis and Dunn's post hoc test. FIG. 3: The body temperature was measured 30 minutes after i.d. injection via transponder read out. Values are means±SEM, n=8-10, a one-way ANOVA and Bonferroni post hoc was used. Asterix indicates a significant difference compared to sham, **** p<0.0001, *** p<0.001, * p<0.05. The ‘&’ sign indicates a significant difference compared to the VitA⁺ group. GFA, short-chain Galacto-oligosaccharides/long-chain Fructo-oligosaccharides/pectin derived Acidic-oligosaccharides; s, standard 4000 IU/kg vitamin A; +, enriched vitamin A 8000 IU/kg.

The acute allergic skin response was significantly different between the sham sensitized mice fed control diet (sham) and whey sensitized mice fed control diet (allergic mice) (p<0.0001) (See FIG. 1).

In addition, all other treatment groups had a significantly higher acute allergic skin response than the sham (p<0.0001) (See FIG. 1). Surprisingly, only whey sensitized mice supplemented with a diet comprising a combination of non-digestible oligosaccharides GFA and enriched levels of Vitamin A (allergic mice fed VitA⁺/GFA) had a significant lower acute allergic skin response compared to the whey sensitized mice supplemented with elevated levels of Vitamin A without GFA (allergic mice fed VitA⁺) (p<0.05). The allergic skin response in the VitA+/GFA group was also lower when compared to the group receiving a diet with GFA and a standard level of vitamin A or the group receiving a diet with standard level of vitamin A and without non-digestible oligosaccharides.

Measured at thirty minutes, the anaphylactic symptom score was significantly higher in the allergic mice, allergic mice fed elevated levels of Vitamin A (VitA⁺) and allergic mice fed non-digestible oligosaccharides GFA and standard level of Vitamin A compared to the sham (p<0.001), while surprisingly this score was not significant higher for the allergic mice fed elevated levels of Vitamin A and non-digestible oligosaccharides GFA (See FIG. 2). Additionally, there was a trend of a lower body temperature (which is indicative for allergic reaction) in the groups with standard levels of vitamin A (with or without the non-digestible oligosaccharides GFA), but this lowering of the body temperature was not observed in the group that had consumed a diet with GFA and enriched levels of Vitamin A (See FIG. 3).

TABLE 1
Anaphylactic shock symptoms at one hour after the
intradermal ear challenge of all mice. Data are
represented in percentages per treated group.

| | | | | % humane |
|---|---|---|---|---|
| | | | % anaphylactic | endpoint |
| | Amount in chow; | | shock (mice | (mice/mice |
| Vitamin A | intake | GFA | total) | with shock) |
| Normal | 4000 IU/kg ≈20 | − | 90 (9/10) | 0 (0/9) |
| | IU/day | + | 80 (8/10) | 0 (0/8) |
| Elevated | 8000 IU/kg; ≈40 | − | 80 (8/10) | 0 (0/8) |
| | IU/day | + | 40 (4/10) | 0 (0/4) |

To measure markers of local T helper cell responses, mRNA levels for Ifnγ (Th1), Il10 and Foxp3 (Treg) were measured in the mid small intestine. For Ifnγ and Foxp3 mRNA the highest levels were observed in the VitA+/GFA group and the increase was statistically significant compared to allergic mice fed VitA+(p<0.05). For Il10 mRNA the same trends were shown. Ifnγ and Foxp3 mRNA expression in allergic mice fed VitA⁺/GFA was also increased compared to sham (p<0.05), and a similar trend was observed for IL-10 mRNA. Foxp3 mRNA was decreased in allergic mice fed an elevated level of Vitamin A compared to allergic mice (p<0.05).

Intracellular cytokine expression of IFNγ was measured in CD11c⁺/CD4⁻ phagocytes of the spleen. Compared to sham, the allergic mice fed VitA⁺/GFA showed an increase in CD11c⁺CD4⁻ IFNγ⁺ phagocytes (p=0.064). In the other groups the level was lower. IFNγ could play a role as an autocrine regulator for DC function. Exogenous IL12 or CD40 ligation combined with high levels of IL18 can induce IFNγ production in immature BALBc derived DC in vitro. One study shows that human monocytes derived from buffy coats pre-treated with IFNγ become regulatory DC and subsequently these IFNγ′ DC produced more IL10 upon stimulation, and expressed more inhibitory molecules and downregulated T cell migration in vitro (Svajger et al. J Leukoc Biol. 2014; 95(1):33-46.7). This suggests that the IFNγ′ phagocytes observed in the spleen could be a more regulatory DC phenotype and the frequency of these cells increases in the mice fed VitA⁺/GFA. This is indicative for induction of immune tolerance.

The results in this example are indicative for increased levels of dietary Vitamin A supporting the non-digestible oligosaccharides to reduce the allergic symptom development in a mouse model for allergic sensitization, and thus of an effect of the combination of dietary vitamin A and non-digestible oligosaccharides on treating and/or preventing allergy, reducing the risk for allergy and inducing and/or enhancing oral immune tolerance to an allergen, in a subject.

### Example 2

Packed powdered Infant Formula, intended for infants suffering from cow's milk allergy, with instructions to reconstitute with water.

After reconstitution the formula, by adding 13.66 g powder to an end volume of 100 ml, the formula comprises 66 kcal per 100 ml. Per 100 ml there is:
3.4 g fat (vegetable fat, fish oil, microbial oil),
7.1 g digestible carbohydrates (mainly lactose and starch),
670 mg non-digestible oligosaccharides (galacto-oligosaccharides and fructo-oligosaccharides in a wt/wt ratio 9/1. Source of galacto-oligosaccharides is VivinalGOS, source of fructo-oligosaccharides is Raftiline HP),
1.6 g protein (extensively hydrolysed whey protein)
100 μg Retinol Equivalent Vitamin A in the form of retinyl palmitate
Minerals, trace elements, other vitamins and micro-ingredients as known in the art and according to directives for infant formula.

### Example 3

Packed powdered Follow On Formula, i) intended for infants that are more prone to developing an allergy or ii) that reduces the risk of developing milk-allergy for infants with a family history of atopy, with instructions to reconstitute with water.

After reconstitution (14.7 g powder to an end volume of 100 ml) the formula comprises 68 kcal per 100 ml. Per 100 ml there is
3.2 g fat (vegetable fat),
8.2 g digestible carbohydrates (mainly lactose and starch),
800 mg non-digestible oligosaccharides (galacto-oligosaccharides and fructo-oligosaccharides in a wt/wt ratio 9/1. Source of galacto-oligosaccharides is VivinalGOS, source of fructo-oligosaccharides is Raftiline HP),
1.6 g protein (partially hydrolysed whey protein)
75 μg Retinol Equivalent Vitamin A in the form of retinyl palmitate
Minerals, trace elements, other vitamins and micro-ingredients as known in the art and according to directives for infant formula.

### Example 4

Packed powdered Infant Formula, for the dietary management of cow's milk allergy, multiple food protein allergies and other where an amino acid based diet is recommended.

The pack contains instructions to reconstitute with water.

After reconstitution, 14.7 g powder to an end volume of 100 ml, the formula comprises 68.2 kcal per 100 ml. Per 100 ml there is
3.4 g fat (mainly vegetable fat),
8.2 g digestible carbohydrates (mainly from glycose syrup),
1.6 g protein equivalent (based on free amino acids)
110 μg RE vitamin A in the form of retinyl acetate,
800 mg non-digestible oligosaccharides (short chain fructo-oligosaccharides and long chain fructo-oligosaccharides in a wt/wt ratio 9/1. Source of short chain fructo-oligosaccharides is Raftilose P95, source of long chain fructo-oligosaccharides in Raftiline HP),
Minerals, trace elements, other vitamins and micro-ingredients as known in the art and according to directives for infant formula

1.-15. (canceled)

16. A method for:
a. treating allergy,
b. preventing allergy,
c. reducing the risk for allergy, and/or
d. inducing and/or enhancing oral immune tolerance to an allergen,
in an infant, the method comprising administering to the infant a combination of
(i) preformed Vitamin A selected from the group consisting of retinol, retinal, retinoic acid and retinyl esters, and
(ii) at least one non-digestible oligosaccharide selected from the group consisting of galacto-oligosaccharide and fructo-oligosaccharides.

17. The method according to claim 16, wherein the combination is in the form of a nutritional composition comprising 5 to 10 μg Retinol Equivalent (RE) of said preformed Vitamin A per gram dry weight and/or 100 to 200 μg RE of said preformed Vitamin A per 100 kcal.

18. The method according to claim 16, wherein the at least one non-digestible oligosaccharide is a mixture of galacto-oligosaccharides and fructo-oligosaccharides.

19. The method according to claim 16, wherein said preformed Vitamin A comprises or are retinyl esters.

20. The method according to claim 16, wherein the subject is at risk of or suffering from a food allergy, in particular a cow's milk protein allergy.

21. The method according to claim 17, wherein the composition comprises 6 to 9 μg RE preformed Vitamin A per g dry weight and/or 120 to 180 μg per 100 kcal.

22. The method according to claim 17, wherein the composition comprises 15 to 250 mg non-digestible oligosaccharides per g dry weight.

23. The method according to claim 17, wherein the composition comprises a source of protein, the protein source being selected from hydrolysed protein or free amino acids.

24. The method according to claim 17, wherein the composition is an infant formula, a follow on formula or a young child formula.

25. The method according to claim 17, wherein said composition comprises two or more non-digestible oligosaccharides selected from the group consisting of galacto-oligosaccharides, fructo-oligosaccharides and uronic acid oligosaccharides.

26. Infant formula, follow on formula or young child formula, comprising:
i. 6 to 9 μg RE preformed Vitamin A per g dry weight and/or 120 to 180 μg RE preformed Vitamin A per 100 kcal, wherein the preformed Vitamin A is selected from the group consisting of retinol, retinal, retinoic acid and retinyl ester,
ii. 15 to 250 mg of at least one non-digestible oligosaccharide selected from the group consisting of galacto-oligosaccharides and fructo-oligosaccharides, per gram dry weight of the formula, and
iii. hydrolysed protein and/or free amino acids.

27. Infant formula, follow on formula or young child formula according to claim 26, wherein the at least one non-digestible oligosaccharide is a mixture of galacto-oligosaccharides and fructo-oligosaccharides.

28. Infant formula, follow on formula or young child formula according to claim 26, comprising two or more non-digestible oligosaccharides selected from the group consisting of galacto-oligosaccharides, fructo-oligosaccharides and uronic acid oligosaccharides.

29. Infant formula, follow on formula or young child formula according to claim 26, wherein the preformed vitamin A is retinol palmitate, and the formula (iii) comprises hydrolysed protein.

30. A method for
a. treating allergy,
b. preventing allergy,
c. reducing the risk of allergy, and/or
d. inducing/enhancing oral immune tolerance to an allergen,
in a subject, wherein the subject is an infant or young child that is at risk of or suffering from a food allergy, wherein the subject is administered with an infant formula, follow on formula or young child formula comprising a combination of (i) and (ii),
wherein the formula comprises,
i. 6 to 9 μg RE preformed Vitamin A per g dry weight and/or 120 to 280 μg RE preformed Vitamin A per 100 kcal, wherein the preformed Vitamin A is selected from the group consisting of retinol, retinal, retinoic acid and retinyl ester,
ii. 15 to 250 mg of at least one non-digestible oligosaccharide selected from the group consisting of galacto-oligosaccharides and fructo-oligosaccharides, per gram dry weight of the formula, and
iii. hydrolysed protein and/or free amino acids.

31. The method according to claim 30, wherein the allergy is a cow's milk protein allergy.

32. The method according to claim 30, wherein the preformed vitamin A is retinol palmitate, and the formula (iii) comprises hydrolysed protein.
